(19) United States
(12) Patent Application Publication
(10) Pub. No.: US 2024/0197256 A1
(43) Pub. Date: Jun. 20, 2024

(54) SMART MATTRESS

(71) Applicant: Seoul National University Hospital, Seoul (KR)

(72) Inventors: Young Ae Song, Seongnam-Si (KR); Hyun Jeong Kim, Yongin-Si (KR)

(21) Appl. No.: 18/270,187

(22) PCT Filed: Dec. 27, 2021
(86) PCT No.: PCT/KR2021/019900

§ 371 Date: Feb. 9, 2024

(30) Foreign Application Priority Data
Dec. 30, 2020 (KR) 10-2020-0187577

(51) Int. Cl. A61B 5/00; A61B 5/0205; A61B 5/024; A61B 5/11; A61B 5/332; A61G 7/018

(52) U.S. Cl. CPC A61B 5/6892; A61B 5/0022; A61B 5/02055; A61B 5/7282; A61G 7/018; A61B 5/02438; A61B 5/1117; A61B 5/332; A61B 2562/0247

(57) ABSTRACT

A smart mattress according to an embodiment may be provided with: an elastic body which elastically supports the weight of a patient; a driving part frame, which supports the elastic body and at least a part of which is deformed to deform the elastic body; a sensor frame disposed under the driving part frame to support the driving part frame; an actuator disposed between the sensor frame and the driving part frame and driven to cause deformation of the driving part frame; a pressure sensor disposed to have a predetermined pattern between the sensor frame and the driving part frame to measure a pressure applied by the patient to the elastic body; at least one sensor for measuring biometric information of the patient; and a control part, wherein the control part drives the actuator on the basis of pressure information measured by the pressure sensor.

### TECHNICAL FIELD

The present disclosure relates to a smart mattress, in more detail, a smart mattress that can monitor a patient by receiving biometric information data of the patient on the basis of Internet of Things (IOT), can make it easy to care a patient in cooperation with a medical information system, and can automatically change the posture of a patient at every predetermined time.

### NATIONAL R&D PROJECT FOR SUPPORTING INVENTION

Subject No.: KMDF_PR_20200901_0102
Department Name: Ministry of Science and ICT, Ministry of Trade, Industry and Energy, Ministry of Health and Welfare, and Ministry of Food and Drug Safety
Project Name: Pan-governmental Lifecycle Medical Device R&D Project
Subject Name: Development and commercialization of IoT-based auto-rotating smart mattress
Research and Management Institution: Korea Medical Device Development Fund
Host Institution: SNUH Bundang Hospital
Research Period: Sep. 1, 2020-Dec. 31, 2023

### BACKGROUND ART

Recently, necessity of the contactless technology for avoiding direct contact between patients and medical staffs in the process of collecting biometric information of patients and treating patients at the medical field is increasing due to COVID-19 Pandemic.

In addition, since one nurse is in charge of 19.5 patients at the medical field, there is a problem that nurses complain of high fatigue due to excessive work. Further, since medical staffs such as a nurse and a caregiver conduct work of assisting activity of patients such as measuring the weight of patients, changing the position of patients, etc., there is a problem that over 70% of the staffs have diseases in the musculoskeletal system.

Accordingly, the need to apply equipment that can assist the work of medical staffs such as a nurse and a caregiver at the medical field is increasing.

Patients spend most time in medical beds in the normal medical environment. However, even though normal medical beds have a mattress, it only provides cushioning to users and do not provide additional functions of being able to assist the work of medical staffs such as monitoring the condition of a patient and changing the position of a patient.

### DETAILED DESCRIPTION OF THE INVENTION

### Technical Problem

The present disclosure has been made in an effort to solve the problems described above, and an objective of the present disclosure is to provide a smart mattress that can collect the biometric information of a patient on the basis of IoT and can change the position of a patient even without contact by a medical staff.

Further, another objective is to provide a system that can systemically apply information collected from a smart mattress to management such as nursing by accumulatively and comprehensively storing and analyzing the information by sharing biometric information collected using the smart mattress with a patient information server such as an EMR.

### Technical Solution

In order to achieve the objectives, a smart mattress according to an embodiment of the present disclosure comprises: an elastic body which elastically supports the weight of a patient; a driving part frame, which supports the elastic body and at least a part of which is deformed to deform the elastic body; a sensor frame disposed under the driving part frame to support the driving part frame; a plurality of actuators disposed between the sensor frame and the driving part frame and driven to cause deformation of the driving part frame; a plurality of pressure sensors disposed to have a predetermined pattern between the sensor frame and the driving part frame to measure a pressure applied by the patient to the elastic body; at least one sensor for measuring biometric information of the patient; and a control part, in which the control part drives at least some of the plurality of actuators on the basis of pressure information measured by the plurality of pressure sensors.

Embodiments of the present disclosure provides also a server that transmits and receives information to and from the smart mattress, for example, a patient information server that transmits and receives information about one or more of biometric signal measurement information measured by the at least one sensor of the smart mattress, pressure information measured by the pressure sensor, and the driving information, and/or a medical management system including the server.

In an embodiment, the server or the control part of the smart mattress can obtain biometric information of a patient by analyzing one or more of a biometric signal detected by the at least one sensor and a pressure signal measured by the pressure sensor using a predetermined algorithm, for example, a machine learning algorithm.

In an embodiment, the smart mattress may further include one or more of: a memory which stores data that are processed in the smart mattress; a communication part including one or more communication modules for enabling the smart mattress to communicate with an external device in a wired or wireless type; and a user interface providing one or more items of information of the state of the smart mattress and the condition of the patient.

In an embodiment, the memory may store one or more of data about an operation pattern of the driving part, an operation time of the driving part, the operation number of times of the driving part, a sleep pattern of the patient, and information obtained from the at least one sensor, the external device may include a terminal that can connect with a wearable device that the patient wears or an EMR system, and the user interface may show one or more of biometric information of the patient, the shape of the mattress, the posture of the patient, and notice information.

In an embodiment, the at least one sensor measuring biometric information of the patient may be at least one sensor selected from a temperature sensor, a pressure sensor, a weight sensor, an electrocardiogram sensor, a heartbeat sensor, and an SpO₂ sensor.

In an embodiment, the temperature sensor may further sense temperature around the smart mattress, posture variation of the patient may be sensed on the basis of information measured by the weight sensor and information measured by the pressure sensor, the electrocardiogram sensor may sense electrocardiogram information before the patient receives treatments on the smart mattress, electrocardiogram information that is changed while the patient receives treatments, and electrocardiogram information after the patient receives treatments, and the heartbeat sensor may sense heartbeat information before the patient receives treatments on the smart mattress, heartbeat information that is changed while the patient receives treatments, and heartbeat information after the patient receives treatments.

In an embodiment, the server or the control part of the smart mattress may set data within a predetermined range of biometric signal data and/or pressure data measured by at least one sensor as normal range data and may set generation of data out of the normal range data as event data.

In an embodiment, the server or the control part of the smart mattress may set the event data as falling event sign data and may provide an alarm signal when received data corresponds to the falling even sign data.

In an embodiment, one mattress may be formed by coupling a plurality of unit or unit body smart mattress modules having the configuration of the smart mattress described above.

In detail, one mattress is formed by coupling a plurality of smart mattress modules that are unit bodies in the smart mattress, and the smart mattress modules may comprise: an elastic body which elastically supports the weight of a patient; a driving part frame, which supports the elastic body and at least a part of which is deformed to deform the elastic body; a sensor frame disposed under the driving part frame to support the driving part frame; a plurality of actuators disposed between the sensor frame and the driving part frame and driven to cause deformation of the driving part frame; and a plurality of pressure sensors disposed to have a predetermined pattern between the sensor frame and the driving part frame for measuring pressure that is applied by the patient to the elastic body.

### Advantageous Effects

The smart mattress according to embodiments of the present disclosure can collect biometric information of a patient in real time and can change the posture of a patient in a contactless manner. Further, it is possible to systematically manage a patient by linking biometric information collected using the smart mattress with a patient information server such as an EMR.

Accordingly, there is an effect that it is possible to construct improved medical system and nursing environment, and it is also possible to improve the health of patient, reduce workload of nurses, improve satisfaction of nurses, and reduce the national medical costs.

### BRIEF DESCRIPTION OF DRAWINGS

FIG. 1 is a block diagram showing the configuration a smart mattress according to an embodiment of the present disclosure.

FIG. 2 is a perspective view of the smart mattress according to an embodiment of the present disclosure.

FIG. 3 is a cross-sectional view taken along line III-III in FIG. 2.

FIG. 4 is view showing pressure recognition by the smart mattress of FIG. 2 and change of posture of a patient by corresponding operation of the smart mattress.

FIG. 5 is a perspective view of a smart mattress according to another embodiment of the present disclosure.

FIG. 6 is a schematic view of a patient management system including a smart mattress according to an embodiment of the present disclosure.

FIGS. 7 and 8 are views showing an example of image information that is displayed on the patient management system of FIG. 6.

### DETAILED DESCRIPTION OF EMBODIMENTS

Hereafter, embodiments of a smart mattress according to examples of the present disclosure are described. Although the present disclosure are described with reference to the embodiments shown in the figures, these are only exemplary embodiments, and the spirit, the main configuration, and application of the present disclosure are not limited by the embodiments.

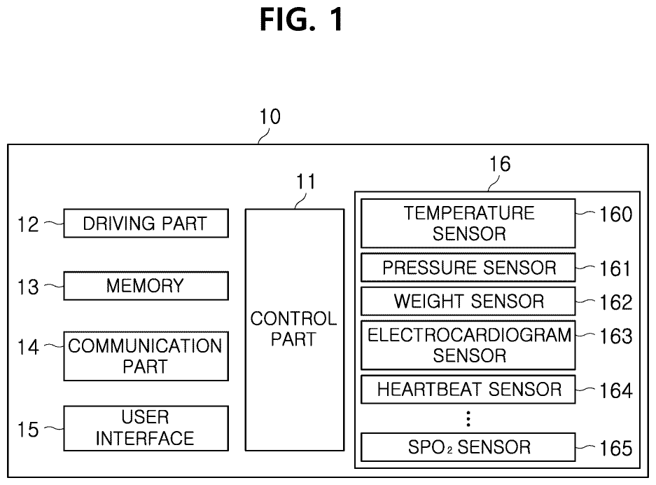

FIG. 1 is a block diagram showing the configuration a smart mattress according to an embodiment of the present disclosure.

Referring to FIG. 1, a smart mattress 10 may include a control part 11, a driving part 12, a memory 13, a communication part 14, a user interface 15, and at least one sensor 16.

The control part 11 generally controls the operation of the smart mattress 10. For example, the control part 11 can control the operation of a driving part 12 that drives the smart mattress 10, which will be described below. Further, the control part 11 can analyze information obtained from at least one sensor 16 of the smart mattress 10, which will be described below, and can control processes to be performed next.

The control part 11 includes at least one processor. The processor may be an array of many logic gates or may be a combination of a common microprocessor and a memory storing programs that can be executed in the microprocessor. Further, those skilled in the art would be able to understand that the processor can be other types of hardware.

The driving part 12 is a component that drives the smart mattress 10. For example, the driving part 12, as will be described below, may include a driving part frame 110 that supports the smart mattress 10 and an actuator 130 that drives the driving part frame 110 to deform at least a portion of the smart mattress 10. The driving part 12 can operate in response to a driving signal from the control part 11. For example, at least a portion of the smart mattress 10 is deformed in accordance with operation of the driving part 12, whereby the posture of a patient P can be changed and the patient P can be prevented from falling from a bed.

The memory 13, which is hardware that stores various data that are processed in the smart mattress 10, can store data that has been processed and will be processed by the control part 11. The memory 13 may be implemented in various types such as a random access memory (RAM), a read-only memory (ROM), and an electrically erasable programmable read-only memory (EEPROM) including a dynamic random access memory (DRAM) and a static random access memory (SRAM).

Various items of information such as data about the operation pattern, operation time, operation number of times of the driving part 12 for operating the smart mattress 10, and a sleep pattern of a patient P, information obtained from the at least one sensor 16, and programs for processing the obtained data can be stored in the memory 13.

The communication part 14 may include one or more modules that enables the smart mattress 10 to communicate with an external device in a wired type or a wireless type. For example, the external device may be a wearable device that a patient P wears or a terminal that can connect with an EMR system.

The communication part 14 may include an internet module part, a near field communication module part, and the like. The internet module part may mean a module for connecting with the internet. The internet module part may be provided inside or outside the smart mattress 10. The near field communication module part is a module for near field communication. The technology of near field communication may be Bluetooth, radio frequency identification (RFID), infrared data association (IrDA), Ultra-Wideband (UWB), ZigBee, etc.

For example, the communication part 14 can receive biometric information of the patient P from a wearable device that a patient P wears, and can transmit the biometric information of the patient P received from the wearable device and/or biometric information of the patient P sensed by the smart mattress 10 to a medical management system including an EMR. Further, the communication part 14 can receive operation signals of the smart mattress 10 from an external device so that the control part 11 can control operation of the smart mattress 10 in accordance with the operation signals.

The user interface 15 can provide information about the state of the smart mattress 10 and/or the condition of a patient P to a user including the patient P and medical staffs. For example, the user interface 15 can show biometric information of the patient P, the shape of the mattress, the posture of the patient, a notice, etc.

The user interface 15 may include various interfacing devices such as a display or a lamp that outputs visual information, a motor that outputs tactile information, a speaker that outputs sound information, terminals for receiving charging power or performing data communication with I/O interfacing devices (e.g., a button or a touch screen) that receives information input from a user or outputs information to a user, and a communication interfacing module for performing wireless communication (e.g., WI-FI, WI-FI Direct, Bluetooth, NFC (Near-Field Communication)) with an external device.

However, only some of the examples of the various user interfaces 15 described above may be selectively employed for the smart mattress 10.

The at least one sensor 16 may include various sensors such as a temperature sensor 160, a pressure sensor 161, a weight sensor 162, an electrocardiogram sensor 163, a heartbeat sensor 164, and an SpO2 sensor 165.

The sensor 16 may include a measurement element that measures variation of predetermined physical quantities and a processor that converts measured physical quantities into digital values and obtains predetermined information from measured physical quantities. The control part 11 may include the processor of the sensor 16. For example, matters related to the predetermined information obtained by the sensor 16 may be applied to the control part 11. The control part 11 can receive a measurement signal from the sensor 16 and obtain predetermined information from the signal.

The temperature sensor 160 can sense the temperature around the smart mattress 10. It is possible to determine whether the temperature in a hospital room is appropriate on the basis of the temperature sensed by the smart mattress 10, and operation of an air conditioner or a heater in a hospital room can be automatically controlled so that the temperature of the hospital room is maintained at an appropriate temperature by linking the air conditioner or the heater with the smart mattress 10 through IoT to be described below.

Further, the temperature sensor 160 may be a sensor that measures the body temperature of a patient P separately from the sensor that senses the temperature around the smart mattress 10. The temperature sensor 160 can figure out whether the body temperature of the patient P is in a normal range by sensing the body temperature of a patient P.

The temperature sensor 160 can measure the temperature around the smart mattress 10 and/or the body temperature of a patient P using various methods known in the art. For example, the temperature sensor 160 may be a thermistor that uses the principle that the resistance of a substrate changes in accordance with temperature. Alternatively, the temperature sensor 160 can measure temperature using thermal expansion of a liquid substance according to temperature. Alternatively, the temperature sensor 160 can measure temperature using electromagnetic waves that are emitted in accordance with surface temperature.

The pressure sensor 161 can measure pressure distribution that is applied by a patient P to the smart mattress 10. For example, the pressure sensor 161 may be a sensor that measures variation of resistance when pressure is applied. The pressure sensor 161 can calculate a pressure value in accordance with such variation of resistance.

As will be described below, the pressure sensors 161 may be scattered in the smart mattress 10. Accordingly, the pressure sensors 161 measure pressure distribution that is applied to the smart mattress 10 in accordance with posture variation of a patient P.

The weight sensor 162 can measure the weight of a patient on the smart mattress 10. For example, the weight sensor 162 may be a load cell that is generally used, and may be installed at each of the corners of a frame supporting the smart mattress 10 or may be installed on the legs of a bed supporting the frame.

The weight sensor 162 can measure weight as biometric information of a patient P. Further, it is possible to sense posture variation of a patient P using the information measured by the weight sensor 162 together with the information measured by the pressure sensor 161.

ECG (Electronic Cardio Graph) is an action current according to contraction and expansion of heart muscles, and the electrocardiogram sensor 163 can measure electrocardiogram information that is an electrical signal related to the electrical activation step of a heart. Action potential that is generated when heart muscles contract and expand generates a current that propagates in the entire body from a heart, the current generates a potential difference in accordance with the position of the body, and the electrocardiogram sensor 163 can measure such potential difference. The electrocardiogram sensor 163 can obtain an impedance value and an electrocardiogram waveform through a contact surface of an electrocardiogram patch. The electrocardiogram sensor 163 can measure the periods of atrium depolarization (P), ventricle depolarization (QRS), and ventricle repolarization (T). The electrocardiogram sensor 163 can figure out whether the electrical activation step of a heart is a standard type on the basis of the maintenance time of each wave, the intervals of waves, the amplitude of each wave, kurtosis, etc.

The electrocardiogram sensor 163 can sense electrocardiogram information of a patient P with electrodes attached to the skin of the patient P when the patient P is positioned on the smart mattress 10. To this end, the electrocardiogram sensor 163 may be provided on the smart mattress 10 or may be provided as equipment separately from the smart mattress 10 to sense electrocardiogram information of a patient P.

The electrocardiogram sensor 163 can continuously sense electrocardiogram information before a patient P receives treatments such as a surgical operation and medication on the smart mattress 10, electrocardiogram information that changes while the patient P receives treatments, and electrocardiogram information after the patient P receives treatments.

The heartbeat sensor 164 can sense heartbeat information of a patient P. The heartbeat information includes a pulse signal obtained by electrically sensing heartbeats.

The heartbeat sensor 164 can be attached to a patient and sense electrocardiogram information of the patient P when the patient P is positioned on the smart mattress 10. To this end, the heartbeat sensor 164 may be provided on the smart mattress 10 or may be provided as equipment separately from the smart mattress 10 and may sense electrocardiogram information of a patient P.

The heartbeat sensor 164 can continuously sense heartbeat information before a patient P receives treatments such as a surgical operation and medication on the smart mattress 10, heartbeat information that changes while the patient P receives treatments, and heartbeat information after the patient P receives treatments.

For example, the heartbeat sensor 164 may be a photo-plethysmography (PPG) sensor. For example, the volume of blood in blood vessels changes due to the pulse, and accordingly, the absorption amount of light changes. The Photo-plethysmography (PPG) sensor can sense variation of such absorption amount of light. The heartbeat sensor 466 is not limited to the photo-plethysmography sensor 466 but may include all of various kinds of sensors that can sense heartbeat information such as heartbeat variability.

Heartbeat information may include a pulse peak interval (PPI), an RR interval, a heart rate, heart rate variability (HRV), pulse transfer time (PTT), pulse wave velocity (PWV), etc.

The pulse peak interval (PPI) is a time interval between continuous peaks. Since the lower the pulse peak interval (PPI), the smaller the time interval between peaks, meaning that the heartbeat is high. Further, since the higher the pulse peak interval (PPI), the larger the time interval between peaks, meaning that the heartbeat is high.

The heart rate variability is information about variation of the pulse peak interval (PPI). The larger the variation of the pulse peak interval (PPI), the larger the heart rate variability, and the smaller the variation of the pulse peak interval (PPI), the smaller the heart rate variability.

The pulse peak interval (PPI) and the heart rate variability may include information about the degree of stress and mental and health conditions of a user. The control part 11 can calculate the degree of stress of a user from measured heartbeat information and heartbeat information.

Oxygen saturation (SpO₂) of blood is the ratio (%) of hemoglobin bonding to oxygen. The SpO₂ sensor 165 can measure the oxygen saturation in the arterial blood of a patient. For example, the SpO₂ sensor 165 can measure the oxygen saturation (SpO₂) in blood in a non-invasive manner by measuring light absorbance for each wavelength by the pulsation component of the arterial blood.

A medical staff can continuously observe the oxygen saturation state of a patient P by quantifying the oxygen saturation state using the SpO₂ sensor 165 of the smart mattress 10 and can quickly cope with a patient who has a possibility of hypoxia.

Meanwhile, the sensor 16 that can be disposed in the smart mattress 10 may further include various sensors, if necessary, without being limited to the kinds described above.

For example, the at least one sensor 16 may further include sensors that can sense various items of biometric information such as the invasive blood pressure (IBP), diastolic blood pressure (NIBP), multigas, brain waves (qCON), breathing, and apnea. For example, a breath sensor can measure whether a patient is breathing in the way of measuring the flow rate of air that is breathed in and out through a nose.

Further, the at least one sensor 160 may further include a fingerprint sensor that can measure fingerprint information from the fingers of a user for user authentication and security, an iris sensor that analysis the iris pattern of an eye of a user, a vein sensor that senses the absorbance of infrared light of reduced hemoglobin in a vein from an image taken from a palm, a face recognition sensor that recognizes characteristic points such as an eye, a nose, a mouth, and facial profile in a 2D or 3D manner, and a radio-frequency identification (RFID) sensor.

Further, the sensor may include a camera, and the camera can analyze and apply face, chest, and abdomen movement of a patient on the smart mattress to breath and heartbeat analysis.

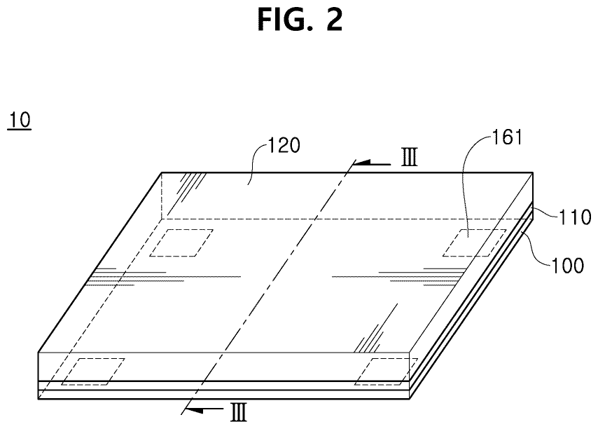

FIG. 2 is a perspective view of the smart mattress according to an embodiment of the present disclosure.

Referring to FIG. 2, the smart mattress 10 includes a sensor frame 100, a driving part frame 110, an elastic body 120, and a pressure sensor 161.

The sensor frame 100 is disposed on the bottom of the smart mattress 10 and supports the smart mattress 10.

A plurality of pressure sensors 161 for measuring pressure that is applied to the smart mattress 10 may be installed on the sensor frame 100. The plurality of pressure sensors 161 is disposed at a plurality of positions to measure pressure distribution throughout the smart mattress 10. In the embodiment shown in FIG. 2, four pressure sensors 161 are disposed at the corners of the smart mattress 10, but this is only for simplifying the drawing and the number and arrangement of the pressure sensors 161 are not limited to those described above. For example, the pressure sensors 161 may be disposed in N×M arrays having predetermined gaps (N and M are arbitrary natural number).

The driving part frame 110 is disposed on the sensor frame 100 and can support the elastic body 120. The driving part frame 110 can be driven by an actuator 130, and accordingly, it is possible to deform at least a portion of the smart mattress 10. Such operation of the driving part frame 110 will be described in detail below.

The elastic body 120, which is a part that supports a patient P in direct contact with the patient P, may be made of a deformable material. The elastic body 120 may be a common bed mattress and may be thin rug such as an underquilt, a mat, and a carpet. Further, the elastic body 120 is easily contaminated because it is a part that directly comes in contact with a patient P. Accordingly, the elastic body 120 can be separated from the smart mattress 10 so that it can be replaced.

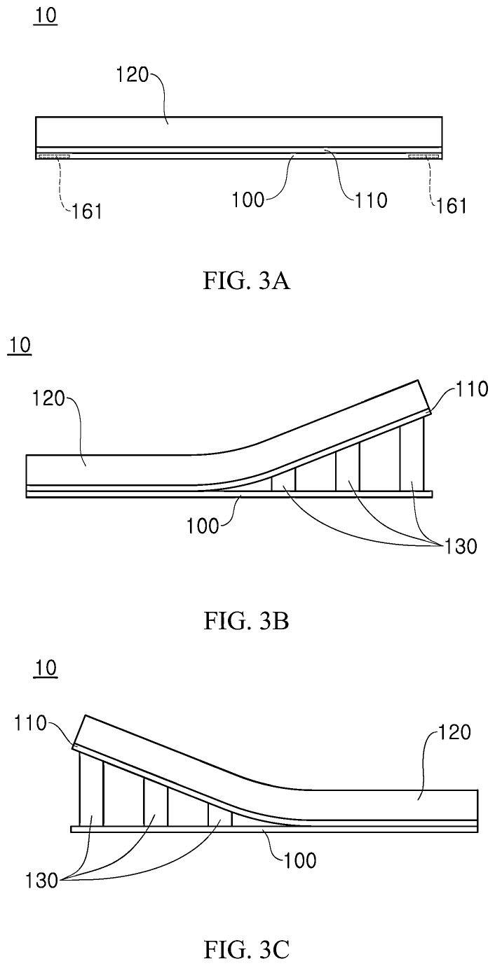

FIG. 3 is a cross-sectional view taken along line III-III in FIG. 2. Hereafter, the configuration of the smart mattress 10 is described on the basis of the left and right of the drawing for the convenience of description.

Referring to FIG. 3, operation of the smart mattress 10 by a driving part 12 is shown. The driving part 12 may include the driving part frame 110 and the actuator 130 that is driven in response to a signal from the control part 11.

The driving part frame 110 includes a left driving part frame and a right driving part frame that can be rotated with respect to the center.

The actuator 130 for driving the driving part frame 110 is disposed between the sensor frame 100 and the driving part frame 110. In the embodiment shown in FIG. 3, three actuators 130 are disposed at the left and three actuators are disposed at the right as well, but the present disclosure is not limited thereto. For example, one actuator 130 may be disposed at each of the left and right. However, by disposing a plurality of actuators 130 at each side, as in the embodiment shown in FIG. 3, it is possible to prevent force being locally applied to the driving part frame 110.

The actuator 130 is disposed on the bottom of the driving part frame 110 and is driven in response to a signal from the control part 11. The left and right driving part frames of the driving part frame 110 can be separately rotated with respect to the center of the driving part frame 110 by the actuator 130. Accordingly, the elastic body 120 disposed on the top of the driving part frame 110 can be deformed in correspondence to rotation of the left and right driving part frames of the driving part frame 110.

For example, (a) of FIG. 3 shows an example of operation of a right actuator. The right driving part frame is rotated counterclockwise by operation of the right actuator, whereby the right part of the elastic body 120 of the smart mattress 10 is deformed. Further, (b) of FIG. 3 shows an example of operation of the left actuator. The left driving part frame is rotated clockwise by operation of the left actuator, whereby the left part of the elastic body 120 of the smart mattress 10 is deformed.

Meanwhile, the driving manner of the driving part frame 110 is not limited to those described above, and various driving manners can be used within a range that is apparent to those skilled in the art. For example, the driving part frame 110 may be directly driven by the actuator 130, as described above, or may be indirectly driven by an actuator. The manner of indirect operation with an actuator means the manner in which an actuator is connected to a power transmission system and drives the driving part frame 110. As an example of the manner of indirect operation with an actuator, the driving part frame 110 may be connected with and driven by a gear assembly that is driven by an actuator. Alternatively, the driving part frame 110 may be supported by a large number of blocks, which can be deformed by an actuator, and can be driven by deformation of at least some of the large number of blocks.

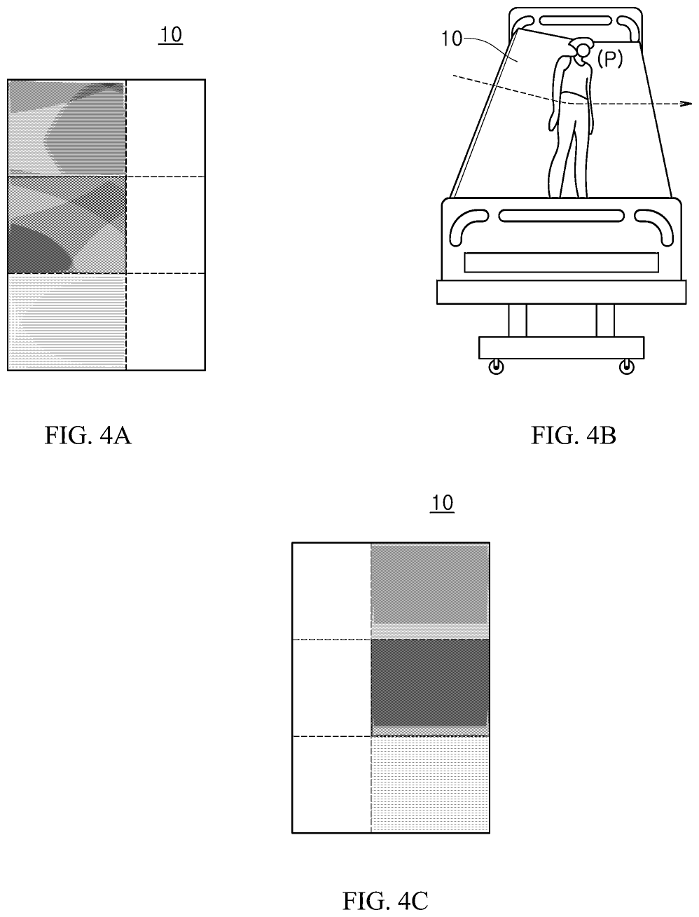

FIG. 4 is a view showing pressure recognition by the smart mattress of FIG. 2 and change of the posture of a patient by corresponding operation of the smart mattress.

(a) of FIG. 4 shows pressure distribution of the smart mattress 10 that is sensed by the pressure sensor 161 of the smart mattress 10. A patient P is lying at the left part on the smart mattress 10, so the pressure sensor 161 disposed at the left part of the smart mattress 10 senses pressure. As described above, since a plurality of pressure sensors 161 may be disposed in the smart mattress 10, it is possible to sense pressure that is applied to each part of the smart mattress 10. Accordingly, the distribution of pressure that is applied to the smart mattress 10 can be shown as in (a) of FIG. 4.

When a patient P is lying in a fixed posture for a long time, pressure is continuously applied to a specific part, so a blood circulation disorder may occur to cause an injury to subcutaneous tissues. This phenomenon is called decubitus ulcer. Accordingly, it is required to change the posture of a patient P at every predetermined time, and to this end, there is a need for labor of a medical staff or a guardian.

When the pressure distribution shown in (a) of FIG. 4 continues for a predetermined time, the smart mattress 10 drives the left part of the smart mattress 10, as shown in (b) of FIG. 4, to change the posture of the patient P. Accordingly, the patient P can be moved while the posture is changed from the left to the right, whereby it is possible to prevent decubitus ulcer of the patient P.

As the smart mattress 10 is driven, as shown in (b) of FIG. 4, the patient P is laid on the right part of the smart mattress 10, and accordingly, the pressure sensor 161 disposed at the right part of the smart mattress 10 senses pressure like the pressure distribution shown in (c) of FIG. 4.

Meanwhile, the pressure distributions of the embodiments shown in (a) and (c) of FIG. 4 are examples and the present disclosure is not limited thereto, and pressure distribution may change in accordance with the posture and body shape of a patient.

Further, the smart mattress 10 may be driven to prevent a patient P from falling from the bed by measuring pressure distribution.

First, the control part 11 of the smart mattress 10 figures out a pressing range and a pressing speed within a normal activity range in which a patient P generally acts, that is, acts without falling.

Next, the control part 11 determines whether there is a danger of falling when the pressing range and the pressing speed change by sensing variation of the pressing range and the pressing speed. Whether there is a danger of falling is determined by comparing the pressing range and the pressing speed within the common activity range of a patient P measured in advance with the pressing range and the pressing speed when the pressing range and the pressing speed change. If the pressing range and the pressing speed when the pressing range and the pressing speed change exceed the allowable range of the pressing range and the pressing speed, the control part 11 operates the driving part frame 110 at the movement direction of a patient P, thereby being able to prevent the patient P from falling from the bed.

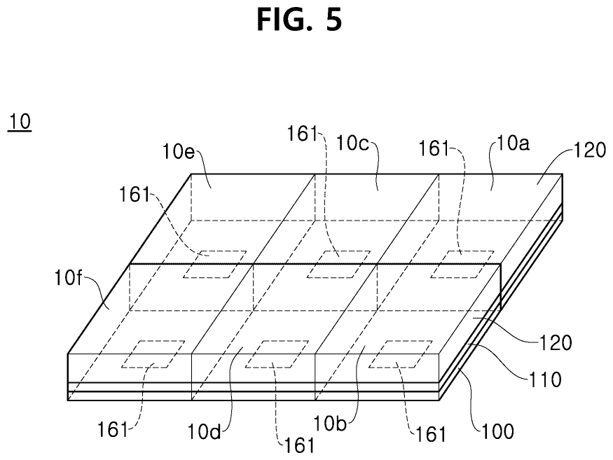

FIG. 5 is a perspective view of a smart mattress according to another embodiment of the present disclosure.

Referring to FIG. 5, a smart mattress 10 may comprise a unit body having the entire or a portion of the configuration of the smart mattress 10 described above. For example, the smart mattress 10 may be modularized, and accordingly, the smart mattress 10 may include a plurality of smart mattress modules 10a to 10f. The smart mattress modules 10a to 10f may be coupled to each other, and when the smart mattress modules 10a to 10f are coupled, they may be electrically connected to each other.

The smart mattress modules 10a to 10f each may include the control part 11, the memory 13, and the communication part 14 described above. Accordingly, the smart mattress modules 10a to 10f each can independently perform the operation of the smart mattress 10 described above. Alternatively, the control part 11, the memory 13, and the communication part 14 may be disposed in the smart mattress 10 separately from the smart mattress modules 10a to 10f. Accordingly, the separate control part 11 may control the operation of each of the smart mattress modules 10a to 10f.

A medical bed on which a patient lives is easy to be contaminated in a common medical environment, and particularly, when contamination is severe, it may be required to replace a mattress. Further, when a mattress is broken as well, it may be required to replace the mattress for repair. Replacing an entire mattress may be accompanied by a cost problem. Further, there is a problem that it is difficult to replace the entire mattresses since mattresses are relatively large in volume.

However, since the smart mattress 10 according to an embodiment of the present disclosure is modularized, when only some of the smart mattress modules 10a to 10f are contaminated or broken, it is possible to replace only the contaminated or broken smart mattress module rather than replacing the entire smart mattress 10. Accordingly, when it is required to replace the smart mattress 10, it is possible to reduce the cost for replacement and improve convenience for replacement as well.

Further, when increasing or decreasing the size of the smart mattress 10 itself, it is possible to easily adjust the size of the smart mattress 10 by additionally coupling or separating the smart mattress modules. Accordingly, it is possible to adjust the size of the smart mattress 10 in accordance with the place and situation in which the smart mattress 10 is disposed, so it is not required to additionally prepare mattresses of other sizes.

Meanwhile, although it is shown in FIG. 5 that the smart mattress 10 includes six smart mattress modules 10a to 10f, the present disclosure is not limited thereto. As described above, it is possible to adjust the number of the smart mattress modules in accordance with the place and situation.

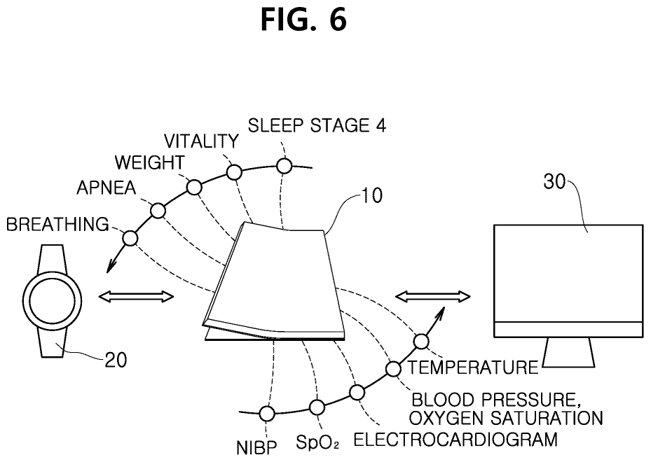

FIG. 6 is a schematic view of a patient management system including a smart mattress according to an embodiment of the present disclosure.

Referring to FIG. 6, as described above, the smart mattress 10 can sense biometric information of a patient P (e.g., the breathing condition, weight, body temperature, blood pressure, oxygen saturation, electrocardiogram, sleep condition, etc. of a patient). The biometric information of a patient P can be sensed by the smart mattress 10. Alternatively, some of the biometric information of a patient P may be sensed by the smart mattress and the others may be sensed by an external device 20 that the patient P wears.

Although a smartwatch that is a kind of wearable devices is shown as the external device 20 in the embodiment shown in FIG. 6, this is just an example and the present disclosure is not limited to that described above. For example, the external device 20 may be not only a smartwatch, but also various devices such as a smartphone that can sense biometric information of a patient P. Further, although only one external device 20 is shown in the embodiment shown in FIG. 5, a plurality of external devices 20 may be provided.

The external device 20 is connected with the smart mattress 10 through an internal network. For example, the smart mattress 10 and the external device 20 may be connected to each other through a gateway. Further, though not shown in FIG. 5, the smart mattress 10 may be connected with several IoT devices through a gateway. Accordingly, a patient P and/or medical staffs can control the operation of several IoT devices through the external device 20 and/or the smart mattress 10.

The internal network described above is a local area network (LAN), and the smart mattress 10, the external device 20, and/or the IoT devices and the gateway can communicate with each other through ZigBee communication, Bluetooth communication, RF443 MHz communication, RF447 MHz communication, Wifi communication, radio frequency identification communication, Ethernet communication, USB communication, serial communication, etc. The communication method between the smart mattress 10 and the external device 20 and/or IoT devices through a gateway is not limited to that described above, and various communication methods may be used in a range that is apparent to those skilled in the art.

Electronic medical record (EMR) means recording and storing all medical records in electronic documents. Accordingly, biometric information of a patient P sensed by the smart mattress 10 and/or the external device 20 should be recorded in a server of the medical management system of a hospital. Accordingly, it is possible to reduce the load on medical staffs and provide improved medical services to patients P by linking the smart mattress 10 with an EMR.

In an embodiment, a medical management system may include a smart mattress 10, a terminal 30, and a server.

The terminal 30 is an electronic device that is used by medical staffs to use or provide a medical management service to customers. For example, the terminal 30 may include all kinds of handheld-based wireless communication devices that can be connected to the server through a network such as a smartphone and a tablet PC. Further, the terminal 30 may include communication devices that can be connected to the server through a network such as a desktop, a tablet PC, a laptop, and an IPTV including a set-top box.

The server is connected with the smart mattress 10 and the terminal 30 through a network and can store biometric information of a patient P sensed by the smart mattress 10. Further, the server can analyze and process the biometric information of the patient P. The biometric information of the patient P analyzed and processed by the server can be provided to medical staffs through the terminal 30.

Though not shown in FIG. 6, the server may include a control part, a communication part, a memory, etc. The control part, the communication part, the memory, etc. of the server may be substantially the same as the control part, the communication part, the memory, etc. of the smart mattress 10 or the external device 20, so the detailed description thereof is omitted.

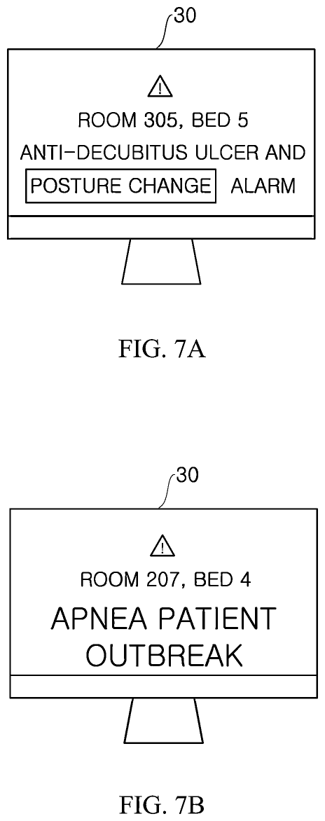

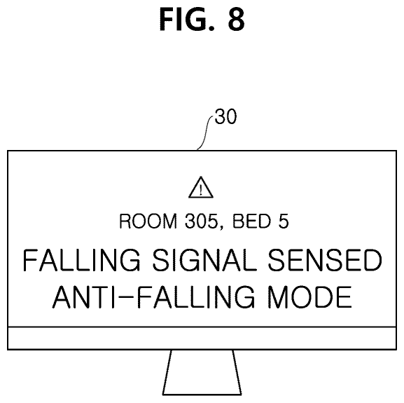

FIGS. 7 and 8 are views showing an example of image information that is displayed on the medical management system of FIG. 6.

Referring to FIG. 7, when it is sensed that predetermined condition variation occurred in a patient P in a hospital room through the smart mattress 10 and/or the external device 20, this information can be provided to medical staffs through the terminal 30.

For example, (a) of FIG. 7 is image information that informs medical staffs that it is required to change the posture of a patient P.

The smart mattress 10 senses distribution of pressure applied on the mattress and the control part 100 determines whether the posture of the patient P has not been changed for a long time by analyzing the pressure distribution. If determining that the patient P has been lying in a fixed posture for a long time, the control part 11 can control the communication part 14 to transmit a notice signal to the medical management system. The communication part 14 transmits a notice signal to the server through the network, and the server receives the notice signal and transmits a notice signal to the terminal 30. The information received from the server is displayed on the terminal, whereby medical staffs can recognize that it is required to change the posture of the patient P.

(b) of FIG. 7 is image information that informs medical staffs that a patient P is in an apnea state.

The smart mattress 10 and/or the external device 20 senses biometric information of the patient P, that is, whether the patient is breathing in the embodiment and the control part 11 determines whether the patient P is normally breathing by analyzing the breath condition of the patient P sensed by the smart mattress 10 and/or the external device 20. If determining that the patient P is not breathing for a predetermined time, the control part 11 can control the communication part 14 to transmit a notice signal to the medical management system. The communication part 14 transmits a notice signal to the server through the network, and the server receives the alarm signal and transmits a notice signal to the terminal 30. The information received from the server is displayed on the terminal, whereby medical staffs can recognize that the patient P is currently in an apnea state. Accordingly, medical staffs can provide medical practice to the patient P.

Meanwhile, in an embodiment, the server or the control part of the smart mattress can set data within a predetermined range of biometric signal data and pressure data measured by at least one sensor as normal range data and can set generation of data out of the normal range data as event data.

Further, in an embodiment, it is possible to set the event data as falling event sign data and provide an alarm signal when received data correspond to falling even sign data.

FIG. 8 shows that the smart mattress 10 is automatically operated in an anti-falling mode when a falling signal is sensed. That is, in an embodiment, when biometric signal data and pressure data sensed by at least one sensor are out of normal range data, that are data in a normal range, they are set as falling event sign data, and when data in a set range are sensed, the actuator 130 of the smart mattress 10 is driven to prevent falling. For example, the smart mattress 10 can prevent falling by inducing a patient P to move to the opposite side by driving the actuator 130.

Meanwhile, in the example described above, analyzing and determining the condition of a patient P are performed by the control part 11 of the smart mattress 11, but the configuration that performs analyzing and determining is not limited to that described above. For example, the smart mattress 10 may transmit only the biometric information of a patient P to a server and the control part of the server may analyze and determine the condition of the patient P on the basis of the biometric information of the patient P.

Although embodiments of the present disclosure were described in more detail with reference to the accompanying drawings, the present disclosure is not limited to the embodiments and may be modified in various ways without departing from the scope of the present disclosure. Accordingly, the embodiments described herein are provided not to limit, but to explain the spirit of the present disclosure, and the spirit of the present disclosure is not limited by the embodiments. Therefore, the embodiments described above are only examples and should not be construed as being limitative in all respects. The protective range of the present disclosure should be construed by the following claims and the scope and spirit of the present disclosure should be construed as being included in the patent right of the present disclosure.

### INDUSTRIAL APPLICABILITY

According to the smart mattresses of embodiments of the present disclosure, it is possible to construct improved medical system and nursing environment, and it is also possible to improve the health of patient, reduce workload of nurses, improve satisfaction of nurses, and reduce the national medical costs.

1. A smart mattress comprising:
an elastic body which elastically supports the weight of a patient;
a driving part frame, which supports the elastic body and at least a part of which is deformed to deform the elastic body;
a sensor frame disposed under the driving part frame and to support the driving part frame;
a plurality of actuators disposed between the sensor frame and the driving part frame and driven to cause deformation of the driving part frame;
a plurality of pressure sensors disposed to have a predetermined pattern between the sensor frame and the driving part frame and to measure a pressure that is applied by the patient to the elastic body;
at least one sensor for measuring biometric information of the patient; and
a control part which drives at least some of the plurality of actuators on the basis of pressure information measured by the plurality of pressure sensors.

2. The smart mattress of claim 1, further comprising at least one of:
a memory which stores data that are processed in the smart mattress;
a communication part including one or more communication modules for enabling the smart mattress to communicate with an external device in a wired or wireless type; and
a user interface providing one or more items of information of the state of the smart mattress and the condition of the patient.

3. The smart mattress of claim 2, wherein one or more of data about an operation pattern of the driving part, an operation time of the driving part, the operation number of times of the driving part, a sleep pattern of the patient, and information obtained from the at least one sensor are stored in the memory,
the external device includes a terminal that can connect with a wearable device that the patient wears or an EMR system, and
the user interface shows one or more of biometric information of the patient, the shape of the mattress, the posture of the patient, and notice information.

4. The smart mattress of claim 1, wherein the at least one sensor measuring biometric information of the patient is at least one sensor selected from a temperature sensor, a pressure sensor, a weight sensor, an electrocardiogram sensor, a heartbeat sensor, and an SpO2 sensor.

5. The smart mattress of claim 4, wherein the temperature sensor further senses temperature around the smart mattress,
posture variation of the patient is sensed on the basis of information measured by the weight sensor and information measured by the pressure sensor,
the electrocardiogram sensor senses electrocardiogram information before the patient receives treatments on the smart mattress, electrocardiogram information that is changed while the patient receives treatments, and electrocardiogram information after the patient receives treatments, and
the heartbeat sensor senses heartbeat information before the patient receives treatments on the smart mattress, heartbeat information that is changed while the patient receives treatments, and heartbeat information after the patient receives treatments.

6. The smart mattress of claim 1, wherein the control part sets data within a predetermined range of biometric signal data measured by the at least one sensor and pressure data measured by the pressure sensor as normal range data and sets generation of data out of the normal range data as event data.

7. The smart mattress of claim 6, wherein the control part sets the event data as falling event sign data, and
provides an alarm signal when one or more of the biometric signal data measured by the at least one sensor and the pressure data measured by the pressure sensor correspond to the falling event sign data.

8. The smart mattress of claim 1, wherein one mattress is formed by coupling a plurality of smart mattress modules that are unit bodies including at least a portion of the configuration of the smart mattress, and
the smart mattress modules include:
an elastic body which elastically supports the weight of a patient;
a driving part frame, which supports the elastic body and at least a part of which is deformed to deform the elastic body;
a sensor frame disposed under the driving part frame to support the driving part frame;
a plurality of actuators disposed between the sensor frame and the driving part frame and driven to cause deformation of the driving part frame; and
a plurality of pressure sensors disposed to have a predetermined pattern between the sensor frame and the driving part frame to measure a pressure that is applied by the patient to the elastic body.

9. A medical management system comprising:
the smart mattress of claim 1; and
a server transmitting and receiving information to and from the smart mattress.
